(19) United States
(12) Patent Application Publication
(10) Pub. No.: US 2011/0046609 A1
(43) Pub. Date: Feb. 24, 2011

(54) INDWELLING CATHETER

(76) Inventor: Michael Notter, Berlin (DE)

Correspondence Address: WARE FRESSOLA VAN DER SLUYS & ADOLPHSON, LLP, BRADFORD GREEN, BUILDING 5, 755 MAIN STREET, P O BOX 224, MONROE, CT, 06468, US

(21) Appl. No.: 12/308,812

(22) PCT Filed: Jun. 22, 2007
(86) PCT No.: PCT/EP2007/056266

§ 371 Date: Dec. 5, 2009

(30) Foreign Application Priority Data
Jun. 22, 2006 (DE) 10 2006 029 574.9

(51) Int. Cl. A61M 39/10; A61M 25/00

(52) U.S. Cl. 604/533; 604/264

(57) ABSTRACT

An indwelling catheter made of a shape memory material with shape memory properties, so that the indwelling catheter comprises a first shape below a transition temperature and a second shape above the transition temperature, wherein the indwelling catheter comprises lamellas separate from one another in circumferential direction at its distal end, which lamellas extend in a longitudinal direction of the catheter below the transition temperature and are bent in outward direction above the transition temperature.

The invention relates to an indwelling catheter with a straight, tubular base body.

The invention relates in particular to an intravenous indwelling catheter for collecting blood components, in particular hemopoietic stem cells through cell separators and for dialysis.

Indwelling catheters have various applications, e.g. already for blood donations or also in the context with dialysis.

Indwelling catheters, which are used for hemopoietic progenitor cell transplantation, after a conditioning therapy, have to meet particular requirements. The hemopoietic progenitor cell transplantation after conditioning chemotherapy with myeloablative or reduced intensity often provides the best healing opportunities for patients with leukemia, lymphoma and plamozytoma. The method requires mobilizing hemopoietic stem cells from the bone marrow into the peripheral blood. This is implemented through chemotherapy in combination with growth factors (G-CSF) for patients or through growth factors alone for allogenic donors. Thereafter, collecting stem cells from the peripheral blood can be implemented by using a blood cell separator similar to the one used for a longer three to four hour blood donation.

Herein, the patient blood is conducted from a venous blood vessel of the patient into the machine, and conducted back to the patient through a second venous access after separation.

The following factors are crucial for the efficiency of a blood collecting method:
Blood flow velocity: It is primarily a function of the inner diameter and the length of the catheter. Optimum flow rates are 8-100 ml/min. At least 30 ml/min are required.
2. Blood flow interruptions: They are a consequence of short term blockages of the distal catheter opening through aspiring the catheter opening against the catheter wall. This phenomenon often becomes apparent as a vibration at the Luer-connection of the catheter. Flow interruptions extend the separation duration.

For a patient or donor with well developed veins, the connection of the venous cycle to the cell separator can be implemented through commercially available dialysis canulas or intravenous indwelling catheters. Though dialysis canulas facilitate high flow rates, they have the disadvantage that they dislocate in particular during longer cell separations and during placement on the inside of a joint, or they can perforate the venous wall. With this respect, plastic catheters have a lower risk, but they cause low blood flow rates and blood flow interruptions at a higher frequency as a consequence of their length, and they comprise higher flow turbulences.

The condition of the veins of a patient deteriorates when the patient has already undergone chemotherapy and steroid therapy treatments. Thus, in approximately 50% of the cases, no peripheral blood accumulation is possible anymore, since the blood flow rates, which can be achieved with venous indwelling catheters, are too low. Additionally, the blood flow is often interrupted through aspiring the venous wall during blood extraction, which delays the establishment of the interface in the cell separator. In this situation, large bore double bore catheters have to be placed in central veins, so that the required flow rates of 80-100 ml/min can be achieved as a prerequisite of a successful stem cell collection. These catheters have inherent risks like bleeding, pneumothorax and air embolism. In rare cases, these incidents are deadly.

Thus, it is the object of the invention to improve indwelling catheters in particular for applications in context of hemopoietic progenitor cell transplantation, but also in the context of the dialysis or the blood donation. It is the object of the invention to improve the donor safety and donor comfort during blood donation. In particular, an improved stem cell collection catheter system shall be provided, which facilitates collecting stem cells through peripheral veins for the majority of patients from whom autologous stem cells shall be collected and for allogenic stem cell donors, and which makes the use of central dialysis catheters redundant. An indwelling catheter shall be provided, which provides improved blood flow properties, which reduces the frequency of blood flow interruptions and which provides the lowest risk possible when puncturing the veins.

Ideally, an indwelling catheter is not limited to an application in the context of collecting stem cells, but can be used for all patients with complicated venous configurations, or with the necessity for longer duration parenteral therapy.

According to the invention, this object is accomplished through an indwelling catheter made from a shape memory material. Such shape memory material facilitates that the indwelling catheter comprises a first shape below a transition temperature and comprises a second shape above the transition temperature. For the indwelling catheter according to the invention, the first shape is defined in that, lamellas at the distal end of the indwelling catheter, which are separated from one another in circumferential direction, extend in longitudinal direction of the catheter below the transition temperature, and bend outward when exceeding the transition temperature, so that a funnel shaped distal catheter opening is provided.

Such a configuration of the distal catheter end ideally comprises several advantages. The blood flow into the catheter is accelerated over a longer distance, and turbulences occurring when the blood enters the catheter are reduced.

Furthermore, the outward bending lamellas spread the vein open in a tent shape and enlarge the blood entry cross section of the catheter. Thereby, in particular aspiring the distal catheter opening against the venous wall is reduced as far as possible, so that blood flow interruptions occur at least less frequently.

Preferably, the shape memory material provides that the indwelling catheter comprises a smaller inner- and outer diameter below the transition temperature than above the transition temperature. Thus, the indwelling catheter in its relative slender state can be inserted into a punctured vein and subsequently expands to a larger diameter. Due to the larger inner diameter, greater blood flow velocities with a lower associated pressure drop become possible.

Suitable inner diameters of the catheter in its first shape below the transition temperature are smaller than 1.5 mm. In its second shape, above the transition temperature, the catheter preferably has an inner diameter of at least 2 mm.

The transition temperature of the form memory material is ideally few degrees below the body temperature, thus e.g. between 28° C. and 35° C. Thus, the indwelling catheter exceeds the transition temperature after inserting into a vein, and consequently assumes its second expanded shape.

Furthermore the indwelling catheter preferably has a length of less a 40 mm and particularly preferably of less than 35 mm. Such small length of the catheter helps reducing flow turbulences. Furthermore such catheter facilitates an inverse puncture, thus a puncture opposite to the direction of the blood flow. The inverse puncture facilitates a blood flow which is not retrograde like in conventional production technique, but orthograde, thus from the front towards the tip of the catheter. The retrograde flow towards the catheter tip means a reversal of the flow direction from the intravenous flow into the intra luminal flow within the catheter.

For a particularly preferred shape of the catheter the lamellas taper at the distal end of the catheter in a distal direction. This means that the measured dimension of the gaps in circumferential direction between the lamellas simultaneously increases between the lamellas from the base of the lamellas to the distal end of the catheter. This means that the lamellas have the largest extension in circumferential direction at their proximal base at which they connect to a tubular proximal catheter which is closed in circumferential direction and that they become more slender towards the distal end of the catheter. A suitable form of the lamellas is e.g. similar to an elongated triangle or trapeze preferably, with a rounded tip of the distal end. The rounded tip prevents that the wall of the blood vessel is injured when the wall is pressed in outward direction through the outward bent lamellas at the distal end of the catheter, so that the blood vessel is expanded.

In the portion of the transition from the proximal end of the lamellas to the distal end of the tubular closed catheter section, thus in the portion of the base of the lamellas, the catheter preferably comprises openings through which a back flow is avoided. Such openings can also be disposed slightly proximal to the base of the lamellas in the distal portion of the proximal catheter section and they are used for enlarging the pass through- or inflow cross section for the inflowing fluid.

The shape memory material of the catheter is preferably plastic material. This way it is possible to maintain the advantages of the plastic catheter in particular the lower risk for injury, without having to deal with the typical disadvantages of a plastic catheter e.g. a small inner diameter.

A suitable polymer as a shape memory material are polymers with shape memory properties in particular multi block polymers. These are in particular:
A material which can be heparinated and which is based on polyurethane and polyamidoamin (PUPA);
A polymer composition comprised of bi-functional isocyanate and/or a trifunctional isocyanate and a polyol with an average Mw=100-500 ratio isocyanate to polyol=(0.9-1.1):1.0;
a homopolymer comprised of lactid or glycolide or a co-polymer made of lactid and glycolides;
a hydroplylic polymer is comprised of one or plural components selected from: poly(ethylen oxide), polyvinyl pyrrolidone, polyvinyl alcohol, poly(ethylenglycol), polyacrylamid, (poly)hydroxyethylmethacrylat, hydrophilic polyurethanes, HYPAN, (poly)hydroxyethylacrylat, hydroxyl ethyl cellulose, hydroxyl propyl cellulose, methoxyl pectin gel, agar, starch, modified starch, alginate, hydroxyl ethyl carbohydrate and mixtures and co-polymers thereof;
Amorphous polyester-urethane networks, namely polymeric networks which can be obtained by transforming hydroxytelechelic pre-polymers, wherein the pre polymers comprise polyester and/or polyether segments with diocynate;
Pre-polymers can also comprise units which are derived from lactic acid caprylacton, dioxanon, glycolic acid, ethyleglycol and/or propolyenglycol.

Examples for non biodegradable polymer segments or polymers with shape memory properties include the following:
Ethylene vinylacetate, poly methyl acid, polyamides, polyethylenes, polyprophylenes, polystyrenes, polyvinylchloride, poly vinyl phenolic, copolymers and mixtures thereof.

Copolymers which include methylacrylic segments as polymer softeners, also show shape memory properties in “dried” state.

A catheter according to the invention facilitates a novel use of such a catheter in the context of producing stem cells and in the context of producing granulocytes, trombocytes or donor lymphocite concentrates and freezing plasmas through aphaeresis. The use of a dwelling catheter made of shape memory plastic in the context of therapeutic aphaeresis is also novel, e.g. for plasma aphaeresis, erythrocyte-exchange or trombocyte depletion and in the context of aphaereses in the context of new cell therapeutic methods e.g. generating dendritic cells, macrophages or lymphocytes. The invention is now shown with described in more detail with reference to the drawing figures, wherein:

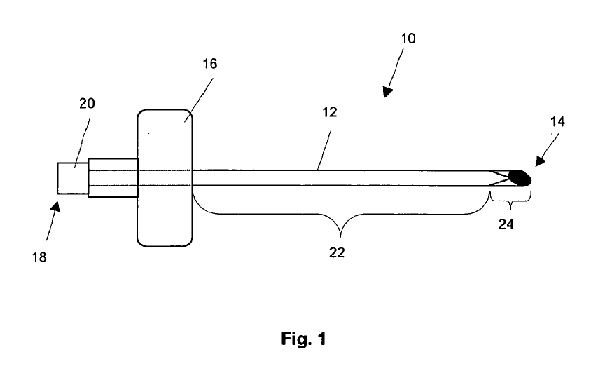

FIG. 1 shows a top view of a catheter according to the invention in an enlarged schematic illustration;

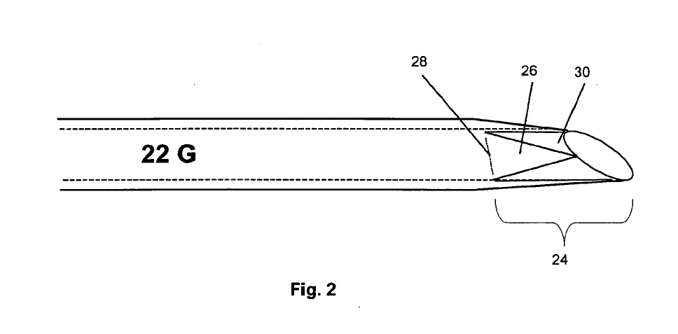

FIG. 2 shows a distal end section of the catheter of FIG. 1 in an enlarged illustration in its first shape below the transition temperature;

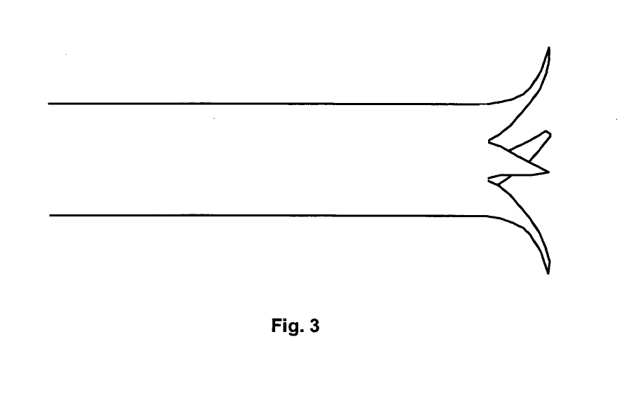

FIG. 3 shows the distal catheter section of FIG. 2 in its second shape above the transition temperature;

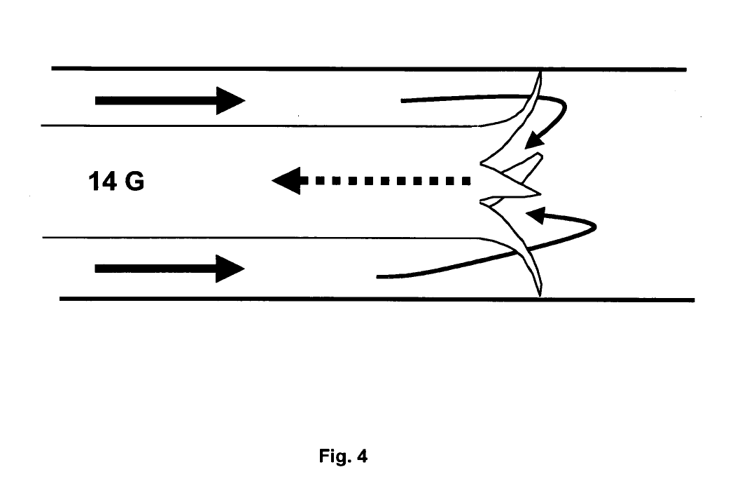

FIG. 4 shows a schematic illustration of the distal catheter section after puncturing in the interior of a blood vessel and after the transition of the catheter shape into a second expanded shape above the transition temperature for a retro grade relative flow; and

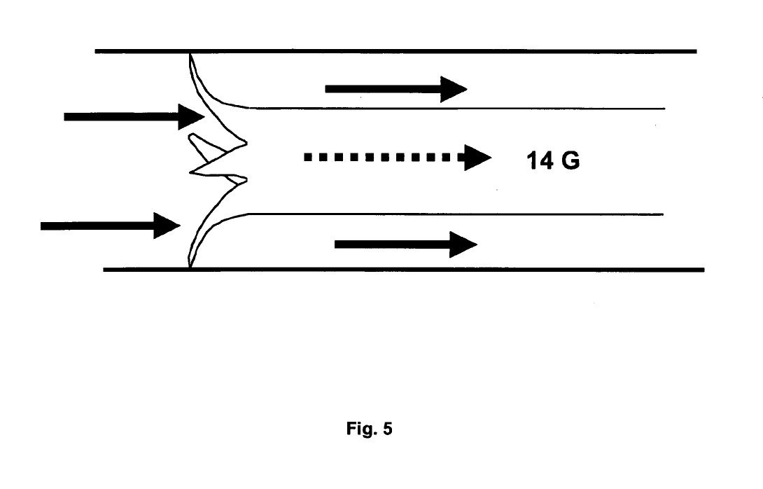

FIG. 5 shows an illustration of a distal catheter section within a blood vessel similar to FIG. 4, but for an orthograde relative flow.

The catheter 10 illustrated in FIG. 1 comprises a straight catheter shaft 12 whose free length from its distal end 14 to a fixation lug 16 close to the proximal catheter end 18 is 3.5 cm. Besides the fixation wing 16, the catheter 10 also comprises a typical Luer connection 20 at is proximal end.

The free straight shaft 12 of the catheter 10 has a relatively long proximal catheter section 22 which is tubular and closed. this proximal catheter section 22 transitions At its distal end into a comparatively short distal catheter section 24, in which the catheter shaft 12 is divided into plural lamellas in circumferential direction.

As can be derived in particular from FIG. 2 the lamellas 26 can have the shape on an elongated triangle which joins the distal end of the tubular closed proximal shaft section 22 with one base 28.

According to the invention, the catheter shaft 12 is comprised of a polymer with shape memory properties, so that the catheter shaft 12 below a transition temperature between 25° C. and 32° C. assumes the first shape illustrated in FIGS. 1 and 2 and changes into the second shape illustrated in FIGS. 3-5 above the transition temperature.

In the first shape of the catheter shaft 12 below the transition temperature the lamellas 26 are e.g. longitudinally extended. In the second shape above the transition temperature the lamellas 26 at the distal end of the catheter shaft 12 are bent outward as shown in FIGS. 3-5.

Furthermore the catheter in its first shape below the transition temperature has an inner diameter of 22 Gauge corresponding approximately to 1 mm. After transitioning into its second shape above the transition temperature, the catheter shaft 12 has an inner diameter of e.g. 14 Gauge, corresponding approximately to 2 mm.

The subsequent advantages can be achieved with such catheter:

Due to the small diameter in its first shape the catheter 10 can be inserted easily into a punctured vein by means of a canule 30 inserted into a lumen of the catheter shaft 12.

Only subsequently, the catheter shaft 12 expands due to the temperature of the blood surrounding it. The canule 30 can be removed easily.

Simultaneously, the lamellas 26 camber outward thus forming a funnel shaped distal catheter opening through which blood can enter easily without being expanded rapidly.

The distal ends of the outward cambered lamellas 26 can thus expand the respective blood vessel, so that on the one hand the flow conditions are improved and on the other hand an aspiration of the wall of the blood vessel is mostly prevented.

Besides the catheter shape shown herein it is also possible to produce a catheter from a polymer with shape memory properties, so that it expands in a manner as shown herein after exceeding the transition temperature, however without comprising the lamellas at the distal end described herein. A catheter made of a polymer with shape memory properties and a shaft which expands when exceeding the transition temperature thus constitutes an invention by itself with can also be implemented with such lamellas, but which causes a synergetic effect with respect to improving flow properties.

What is claimed is:

1. An indwelling catheter made of a shape memory material with shape memory properties, so that the indwelling catheter comprises a first shape below a transition temperature and a second shape above the transition temperature, wherein the indwelling catheter comprises lamellas separate from one another in circumferential direction at its distal end, which lamellas extend in a longitudinal direction of the catheter below the transition temperature and are bent in outward direction above the transition temperature.

2. An indwelling catheter according to claim 1, wherein the indwelling catheter comprises a smaller inner diameter below the transition temperature than above the transition temperature.

3. An indwelling catheter according to claim 2 wherein the inner diameter above the transition temperature is greater than 1.5 mm.

4. An indwelling catheter according to claim 3, wherein the inner diameter above the transition temperature greater than 2.0 mm.

5. An indwelling catheter according to one of the claims 1 through 4, wherein the length of the catheter is less than 40 mm.

6. An indwelling catheter according to one of the claims 1 through 5, wherein the proximal catheter section in the portion of the transition to the proximal end of the lamellas comprises openings which prevent reverse flow.

7. An indwelling catheter according to one of the claims 1 through 6, wherein the lamellas respectively have the shape of a triangle, whose base joins a proximal catheter section which is closed in a tubular shape in circumferential direction and whose tip respectively forms a distal end of the catheter.

8. An indwelling catheter according to one of the claims 1 through 7, wherein the catheter comprises a Luer connection at its proximal end.

9. An indwelling catheter according to one of the claims 1 through 8, wherein the shape memory material is a polymer material.

10. An indwelling catheter according to one of the claims 1 through 9, wherein, the transition temperature of the shape memory material is between 28° C. and 35° C.
